(19) United States
(12) Patent Application Publication
(10) Pub. No.: US 2011/0306762 A1
(43) Pub. Date: Dec. 15, 2011

(54) ACID ADDITION SALT OF UDENAFIL, PREPARATION METHOD THEREOF AND PHARMACEUTICAL COMPOSITION COMPRISING THE SAME

(75) Inventors: Chan-Ho Lee, Gyeonggi-do (KR); Chang-Yong Shin, Seoul (KR); Seul-Min Choi, Gyeonggi-do (KR); Kyung-Koo Kang, Gyeonggi-do (KR); Dong-Seong Kim, Seoul (KR); Byoung-Ok Ahn, Gyeonggi-do (KR); Moo-Hi Yoo, Seoul (KR)

(73) Assignee: Dong-A Pharm. Co., Ltd., Seoul (KR)

(21) Appl. No.: 13/201,900

(22) PCT Filed: Feb. 17, 2010
(86) PCT No.: PCT/KR10/00976

§ 371 Date: Aug. 17, 2011

(30) Foreign Application Priority Data
Feb. 18, 2009 (KR) 10-2009-0013350

(51) Int. Cl. C07D 487/04

(52) U.S. Cl. 544/262

(57) ABSTRACT

The present invention provides an acid addition salt of Udenafil, a preparation method thereof and a pharmaceutical composition comprising the same. The acid addition salt of Udenafil in which Udenafil is bonded to an organic acid selected from the group consisting of oxalic acid, benzenesulfonic acid, camphorsulfonic acid, cinnamic acid, adipic acid and cyclamic acid, has excellent solubility in an aqueous medium, water stability and crystallinity, thereby being suitably applied for a pharmaceutical composition.

### FIELD OF THE INVENTION

The present invention relates to an acid addition salt of Udenafil, a preparation method thereof and a pharmaceutical composition comprising the same.

More specifically, the present invention relates to an acid addition salt of Udenafil which is a crystalline form having good solubility in an aqueous medium and excellent stability, especially, outstanding stability to water, a preparation method thereof and a pharmaceutical composition comprising the same.

### BACKGROUND OF THE INVENTION

5-[2-propyloxy-5-(1-methyl-2-pyrrolidinyleneamidosulfonyl)phenyl]-1-methyl-propyl-1,6-dihydro-7H-pyrazolo(4,3-d)pyrimidine-7-one [‘Udenafil’ (WO 00/027848, Korean Patent No. 0353014)] represented by the following Chemical formula 1 is an PDE-5 inhibitor. It has been reported that the compound has notably higher selectivity to PDE-5 than the conventional compounds and shows decreased side effect:

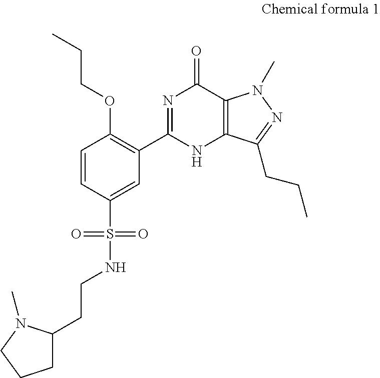

In addition, It has been known that since the maximum peak plasma concentration of Udenafil is reached in 1 hour and its half-life is 12 hours, its efficacy can be showed rapidly and be maintained for a long time in vivo. Thus, it has a merit to be administered only once in a day.

However, Udenafil has 6.5 of pKa1 and 12.5 of pKa2 and is in the form of white or yellowish-white powder but not hydrate and solvate. Thus, it has low solubility in an aqueous medium such as water.

The solubility of drug is one of various factors affecting the drug absorption. The dissolution of drug in an aqueous medium is an important pre-step of the absorption in the whole body. Particularly, because the gastro-intestinal dissolution rate of a drug having low water solubility is a rate-limiting factor of the systemic absorption, the bioavailability of the drug can be determined by dissolution test result. Therefore, the drug needs to have appropriate solubility in an aqueous medium in order to show preferred bioavailability and treatment effect. An insoluble compound with low solubility shows irregular absorption and it cannot be expected to provide efficient treatment effect. A drug having low solubility in gastro-intestinal tract cannot be absorbed completely and needs to be formulated in a special formulation form. Accordingly, to efficiently express the drug efficacy, the solubility of water-insoluble Udenafil needs to be improved.

KR 1987-0009998 discloses that a pharmaceutically-acceptable salt of a drug should satisfy the physiochemical properties of excellent stability, non-hygroscopicity, workability for formulating tablet, besides excellent solubility. In order that a salt of an active ingredient is usefully applied for a pharmaceutical composition, it needs to show good stability, crystallinity required for long-term storage, and stability to water which causes hydrolysis and chemical decomposition.

### SUMMARY OF THE INVENTION

The present invention provides acid addition salt of Udenafil which has excellent solubility in an aqueous medium, water stability and crystallinity, and can be suitably applied for a pharmaceutical composition.

Further, the present invention provides a preparing method of the acid addition salt of Udenafil.

Furthermore, the present invention provides a pharmaceutical composition comprising the acid addition salt of Udenafil.

### DETAILED DESCRIPTION OF THE EMBODIMENTS

An embodiment of the present invention provides an acid addition salt of Udenafil in which Udenafil is bonded to an organic acid selected from the group consisting of oxalic acid, benzenesulfonic acid, camphorsulfonic acid, cinnamic acid, adipic acid and cyclamic acid.

In addition, another embodiment of the present invention provides a method of preparing an acid addition salt of Udenafil comprising a step of reacting Udenafil with an organic acid selected from the group of consisting of oxalic acid, benzenesulfonic acid, camphorsulfonic acid, cinnamic acid, adipic acid and cyclamic acid.

Further embodiment of the present invention provides a pharmaceutical composition comprising an acid addition salt according to an embodiment of the present invention as an active ingredient for use of prevention or treatment of impotence, portal hypertension, pulmonary hypertension, benign prostatic hyperplasia associated with lower urinary tract symptom, heart failure or chronic obstructive pulmonary disease.

Hereinafter, an acid addition salt of Udenafil, a preparation method thereof and a pharmaceutical composition comprising the same according to the specific embodiments of the present invention will be explained in detail.

According to an embodiment, an acid addition salt of Udenafil in which Udenafil is bonded to an organic acid selected from the group consisting of oxalic acid, benzenesulfonic acid, camphorsulfonic acid, cinnamic acid, adipic acid and cyclamic acid is provided.

As experimental results, the present inventors found that an acid addition salt of Udenafil such as Udenafil oxalate, Udenafil adipiate, Udenafil benzenesulfonate, Udenafil camphorsulfonate, Udenafil cinnamate and Udenafil cyclamate, can be easily crystallized, and has low hygroscopicity and excellent stability (particularly, water stability), compared with other salts of Udenafil, thereby being suitably applied for a pharmaceutical composition. While Udenafil or other salts of Udenafil are rarely dissolved in an aqueous medium, the acid addition salt of Udenafil according to an embodiment has remarkably high solubility in the aqueous medium such as water.

Accordingly, the specific acid addition salt of Udenafil according to the embodiment has improved bioavailability and thus shows good treatment efficacy. Moreover, the specific acid addition salt of Udenafil has excellent stability (particularly, water stability) due to the crystallinity, long-term storage, and workability for formulation, thereby providing a good active ingredient for a pharmaceutical composition.

Among the examples of the acid addition salt of Udenafil according to the embodiment, Udenafil adipiate, camphorsulfonate and oxalate can be preferably used. As experimental results, the present inventors confirmed that Udenafil adipiate, camphorsulfonate and oxalate showed superior solubility in an aqueous medium than other acid addition salts, and achieved better bioavailability than Udenafil itself. More particularly, Udenafil adipiate shows most excellent solubility among the three acid addition salts, thus can be more preferably used.

The acid addition salt of Udenafil according to the embodiment may be represented by the following Chemical formula 2:

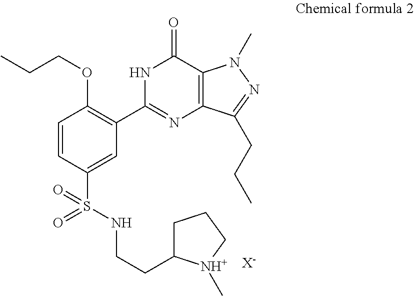

In the formula 2, X⁻ is

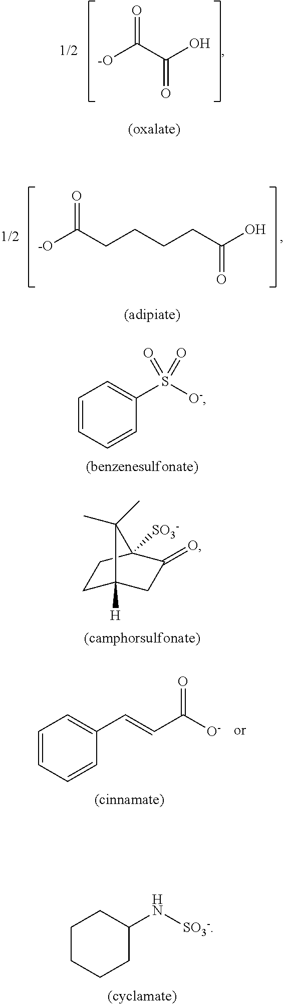

Referring to Chemical formula 2, the acid addition salt of Udenafil has a chemical structure that amine group in the specific site of Udenafil is bonded to organic acids of oxalic acid, benzenesulfonic acid, camphorsulfonic acid, cinnamic acid, adipic acid or cyclamic acid. The acid addition salt of Udenafil is easily crystallized and formulated, shows excellent stability (particularly, excellent water stability), and has good solubility in an aqueous medium, and can be stored for a long time.

Accordingly, the acid addition salt of Udenafil can be suitably used as an active ingredient for a pharmaceutical composition with excellent treatment efficacy due to good bioavailability as well as superior stability.

According to another embodiment, a method of preparing the acid addition salt of Udenafil is provided. The method of preparing the acid addition salt of Udenafil comprises a step of reacting Udenafil with an organic acid selected from the group consisting of oxalic acid, benzenesulfonic acid, camphorsulfonic acid, cinnamic acid, adipic acid and cyclamic acid.

The method of preparing the acid addition salt of Udenafil is described in detail.

Firstly, Udenafil of the following Chemical formula 1, which is used as a starting material for preparing the acid addition salt of Udenafil, can be prepared by performing the following three steps as described in WO 00/027848.

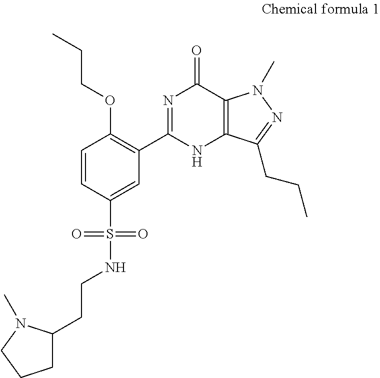

As a first step, 4-[2-propyloxy-5-(chlorosulfonyl)benzamido]-1-methyl-3-propyl-5-carbamoyl pyrazole is prepared by reacting 4-[2-propyloxybenzamido]-1-methyl-3-propyl-5-carbamoyl pyrazole with chlorosulfonic acid.

In the second step, the compound prepared in the first step is reacted with 2-(2-aminoethyl)-1-methyl pyrrolidine to produce 4-[2-propyloxy-5-(1-methyl-2-pyrrolidinylethylamidosulfonyl)benzamido]-1-methyl-3-propyl-5-carbamoyl pyrazole. The reaction can be carried out in a solvent such as chloromethane.

In the third step, the compound obtained in the second step is dissolved in a solvent such as t-butanol, and potassium t-butoxide is added in the solvent. Thus, the compound of Chemical formula 1 is obtained

According to another embodiment, the acid addition salt of Udenafil is obtained by reacting Udenafil, prepared by the above steps, with one organic acid selected from the group consisting of oxalic acid, benzenesulfonic acid, camphorsulfonic acid, cinnamic acid, adipic acid and cyclamic acid. The reaction process can be provided as the following reaction scheme 1.

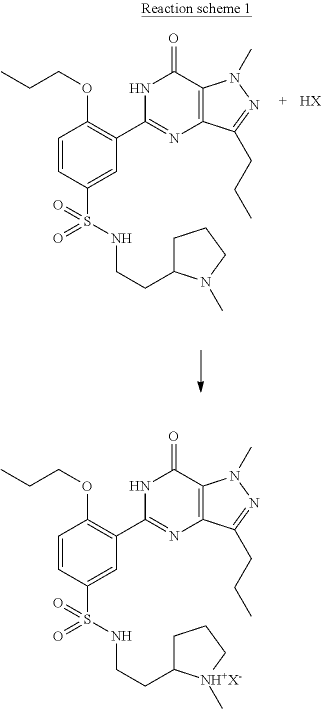

In the above reaction scheme 1, HX is oxalic acid, benzenesulfonic acid, camphorsulfonic acid, cinnamic acid, adipic acid or cyclamic acid, and X⁻ is

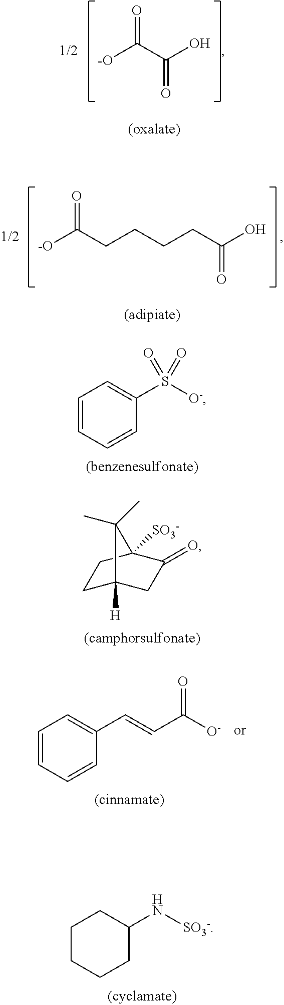

The reaction of Udenafil and the organic acid can be carried out by dissolving the reacting materials in any organic solvent which is capable of performing acid-base reaction.

To obtain the acid addition salt of Udenafil in which Udenafil is bonded to the organic acid in an appropriate equivalent ratio by performing the acid-base reaction suitably, the reaction of Udenafil and the organic acid can be carried out in at least one solvent selected from the group consisting of acetone, ethylacetate, methanol, ethanol, tetrahydrofuran and acetonitrile.

In addition, to carry out the reaction of Udenafil and the organic acid, equivalent ratio of the organic acid to Udenafil may be 0.9 to 2 equivalents, and preferably 0.95 to 1.1 equivalents. By reacting Udenafil and the organic acid in the equivalent ratio, preferable acid addition salt of Udenafil can be obtained.

The reaction of Udenafil and the organic acid can be carried out in the solvent at −5 to 100° C., and preferably 0 to 80° C., for 1 to 24 hours, and preferably 1 to 3 hours.

According to still further embodiment, a pharmaceutical composition comprising the acid addition salt of Udenafil is provided. The pharmaceutical composition comprises the acid addition salt of Udenafil according to an embodiment as an active ingredient and is used for prevention or treatment of impotence, portal hypertension, pulmonary hypertension, benign prostatic hyperplasia associated with lower urinary tract symptom, heart failure or chronic obstructive pulmonary disease.

Udenafil has been reported to be used for treating male impotence, which is one of male sexual dysfunction [WO 00/027848 and Korean Patent No. 0353014]. Udenafil is also used for preventing and treating benign prostatic hyperplasia (BPH) and lower urinary tract symptom (LUTS), and is used as an relaxants of urinary tract smooth muscle or prostatic smooth muscle (Korean laid-open publication no. 2006-0030724).

Further, Udenafil inhibits phosphodiesteras V (PDE-5) enzyme catalyzing the intracellular degradation of cyclic guanosine monophosphatase (cGMP). Thus, Udenafil suppresses the enlargement of left ventricle and the reduction in ventricular wall thickness induced by chronic heart failure, and also inhibits the increase of atrial natriuretic peptide (ANP) in heart tissue and blood and the ventricle fibrosis. Accordingly, Udenafil has been reported to be used for treatment of chronic heart failure (KR 2008-0108185A).

In addition, Udenafil has been reported to be also used for treating and preventing portal hypertension (WO 06/132460 and Korean laid-open publication no. 2006-0030724), hypertension such as pulmonary hypertension, and pulmonary disease such as chronic obstructive pulmonary disease, due to the inhibition activity to PDE-5 enzyme.

Thus, the pharmaceutical composition according to the embodiment is suitably used for prevention and treatment of impotence, portal hypertension, hypertension such as pulmonary hypertension, lower urinary tract symptom, benign prostatic hyperplasia associated with lower urinary tract symptom, heart failure such as chronic heart failure, and pulmonary disease such as chronic obstructive pulmonary disease.

In particular, the pharmaceutical composition comprises an active ingredient of the specific acid addition salt of Udenafil having superior solubility, improved bioavailability, excellent stability (particularly, water stability) and workability for formulation. Thus, the pharmaceutical composition is preferably used for prevention and treatment of the above diseases.

Meanwhile, the pharmaceutical composition may be formulated in a wide variety of oral or parenteral dosage forms on clinical application. Each of the dosage forms may contain various disintegrating agents, surfactants, fillers, thickeners, binders, diluents such as wetting agents or other pharmaceutically acceptable excipients.

For example, the pharmaceutical composition may be formulated in a solid dosage form for oral administration, and the solid dosage form may be powders, granules, capsules, tablets or pills. Further, the solid dosage form may include one or more excipients such as calcium carbonate, starch, sucrose, lactose, microcrystalline cellulose or gelatin. In addition, the solid dosage form may include, in addition to the excipients, a lubricant such as talc or magnesium stearate.

Also, the pharmaceutical composition may be formulated in a liquid dosage form for oral administration, and the solid dosage form may be suspensions, emulsions or syrups. Further, the liquid dosage form may include, in addition to commonly used simple diluents such as water and liquid paraffin, various excipients such as humectants, sweeteners, aromatics or preservatives.

In addition, the pharmaceutical composition may be formulated in a dosage form for parenteral administration, and the dosage form may be sterile aqueous solutions, suspensions, emulsions, non-aqueous solutions or suppositories. More specifically, the non-aqueous solutions or suspensions may include propyleneglycol, polyethyleneglycol, vegetable oils such as olive oil or injectable esters such as ethyl oleate. As a base for suppositories, witepsol, macrogol, tween 61, cacao oil, laurin oil or glycerinated gelatin may be used.

Meanwhile, the dosage of the pharmaceutical composition may vary depending on the patient's weight, age, gender, administration time and mode, excretion rate, and the severity of disease. For example, a dosage unit of the pharmaceutical composition, that is administered at one time, may include 10 to 100 mg, and preferably 10 to 50 mg of the active ingredient (i.e., the acid addition salt of Udenafil), and the dosage unit may be administered to an adult once or several-times daily.

### EXAMPLES

The invention is further described and illustrated in examples provided below, which are, however, not intended to limit the scope of the invention.

### Example 1

### Preparation of an Oxalic Acid Addition Salt of Udenafil

1 g of Udenafil was suspended in 10 mL of ethanol and agitated at room temperature. 0.25 g (1 equivalent) of oxalic acid was added dropwise into the reaction solution. After agitating the reaction solution for 1 hour at room temperature, the produced solid was filtered, washed with n-hexane 5 ml, and dried in vacuum. Consequently, white crystal of the title compound 1.05 g was obtained with a yield of 89.5%.

1H-NMR (DMSO-d6): 0.94 (m, 6H), 1.59 (m, 2H), 1.62 (m, 4H), 1.85 (m, 2H), 1.96 (m, 1H), 2.13 (m, 1H), 2.69 (s, 3H), 2.85 (m, 2H), 2.95 (m, 1H), 3.15 (m, 1H), 4.08 (t, 2H), 4.15 (s, 3H), 7.33 (d, 1H), 7.76 (bs, 1H), 7.87 (d, 1H), 7.93 (s, 1H), 12.16 (s, 1H)

### Example 2

### Preparation of a Benzenesulfonic Acid Addition Salt of Udenafil

1 g of Udenafil was suspended in 10 mL of acetonitrile and 1 mL of methanol and agitated at room temperature. 0.31 g (1 equivalent) of benzenesulfonic acid was added dropwise into the reaction solution. After agitating the reaction solution for 1 hour at 80° C. and then 1 hour at 50° C., the produced solid was filtered, washed with acetone 5 mL, and dried in vacuum. Consequently, white crystal of the title compound 0.98 g was obtained with a yield of 74.8%.

1H-NMR (DMSO-d6): 0.93 (m, 6H), 1.60 (m, 2H), 1.70 (m, 4H), 1.84 (m, 1H), 1.97 (m, 2H), 2.18 (m, 1H), 2.81 (m, 7H), 3.05 (m, 1H), 3.25 (m, 1H), 3.53 (m, 1H), 4.09 (t, 2H), 4.15 (s, 3H), 7.32 (m, 2H), 7.57 (d, 1H), 7.76 (t, 1H), 7.87 (d, 1H), 7.94 (s, 1H), 9.30 (bs, 1H), 12.15 (s, 1H)

### Example 3

### Preparation of a Camphorsulfonic Acid Addition Salt of Udenafil

1 g of Udenafil was suspended in 20 mL of ethylacetate and agitated at room temperature. 0.45 g (1 equivalent) of camphorsulfonic acid was added dropwise into the reaction solution. After agitating the reaction solution for 3 hour at room temperature, the produced solid was filtered, washed with ethylacetate 10 ml, and dried in vacuum. Consequently, white crystal of the title compound 1.23 g was obtained with a yield of 89.4%.

1H-NMR (DMSO-d6): 0.72 (s, 3H), 0.92 (m, 6H), 1.02 (s, 3H), 1.24 (m, 2H), 1.57 (m, 2H), 1.70 (m, 2H), 1.82 (m, 3H), 1.96 (m, 3H), 2.20 (m, 2H), 2.35 (d, 1H), 3.05 (m, 1H), 3.26 (m, 1H), 3.55 (m, 1H), 4.07 (t, 2H), 4.14 (s, 3H), 7.34 (d, 1H), 7.75 (t, 1H), 7.87 (d, 1H), 9.35 (bs, 1H), 12.14 (s, 1H)

### Example 4

### Preparation of a Cinnamic Acid Addition Salt of Udenafil

1 g of Udenafil was suspended in 10 mL of tetrahydrofuran and agitated at room temperature. 0.29 g (1 equivalent) of cinnamic acid was added dropwise into the reaction solution. After agitating the reaction solution for 3 hour at room temperature, the produced solid was filtered, washed with acetone 5 ml, and dried in vacuum. Consequently, white crystal of the title compound 1.02 g was obtained with a yield of 79.1%.

1H-NMR (DMSO-d6): 0.92 (m, 6H), 1.27 (m, 2H), 1.54 (m, 2H), 1.67 (m, 5H), 1.99 (m, 2H), 2.12 (s, 3H), 2.75 (m, 3H), 2.86 (m, 1H), 3.59 (t, 2H), 4.08 (t, 2H), 4.15 (s, 3H), 6.52 (d, 1H), 7.33 (d, 1H), 7.40 (m, 3H), 7.55 (d, 1H), 7.66 (m, 2H), 7.85 (d, 1H), 7.94 (s, 1H), 12.16 (bs, 1H)<

### Example 5

### Preparation of an Adipic Acid Addition Salt of Udenafil

1 g of Udenafil was suspended in 10 mL of acetone and agitated at room temperature. 0.28 g (1 equivalent) of adipic acid was added dropwise into the reaction solution. After agitating the reaction solution for 3 hour at room temperature, the produced solid was filtered, washed with acetone 5 ml, and dried in vacuum. Consequently, white crystal of the title compound 1.16 g was obtained with a yield of 90.6%.

1H-NMR (DMSO-d6): 0.97 (m, 6H), 1.27 (m, 2H), 1.52 (m, 6H), 1.78 (m, 6H), 1.99 (m, 3H), 2.11 (s, 3H), 2.18 (m, 3H), 2.75 (m, 4H), 2.80 (m, 1H), 4.06 (t, 2H), 4.15 (s, 3H), 7.32 (d, 1H), 7.86 (d, 1H), 7.93 (s, 1H), 12.09 (bs, 1H)

### Example 6

### Preparation of a Cyclamic Acid Addition Salt of Udenafil

1 g of Udenafil was suspended in 10 mL of ethylacetate and agitated at room temperature. 0.35 g (1 equivalent) of cyclamic acid was added dropwise into the reaction solution. After agitating the reaction solution for 3 hour at room temperature, the produced solid was filtered, washed with ethylacetate 5 ml, and dried in vacuum. Consequently, white crystal of the title compound 1.13 g was obtained with a yield of 83.7%.

1H-NMR (DMSO-d6): 0.93 (m, 6H), 1.03 (m, 3H), 1.16 (m, 2H), 1.36 (m, 5H), 1.77 (m, 4H), 1.89 (m, 5H), 2.13 (m, 1H), 2.70 (m, 3H), 2.78 (t, 3H), 2.84 (m, 3H), 3.13 (m, 1H), 3.46 (m, 1H), 4.07 (t, 2H), 4.14 (s, 3H), 7.34 (d, 1H), 7.73 (s, 1H), 7.87 (d, 1H), 7.94 (s, 1H), 12.13 (s, 1H)

### Comparative Example

### Preparation of Acid Addition Salts of Udenafil Bonded to Fumaric acid, maleic acid, aspartic acid, glutamic acid, citric acid, succinic acid, Hippuric Acid, Tartaric Acid, Lactic Acid, Maleic Acid, Malonic Acid, Glutaric Acid or Formic Acid

Except that fumaric acid, maleic acid, aspartic acid, glutamic acid, citric acid, succinic acid, hippuric acid, tartaric acid, lactic acid, maleic acid, malonic acid, glutaric acid or formic acid was used instead of organic acids used in Examples 1 to 6, the preparation method was substantially same as Examples 1 to 6 to produce the acid addition salts of Udenafil.

In these comparative examples, acid addition salts of Udenafil in crystalline form could not be obtained. Some organic acids were not dissolved in the organic solvents and thus acid addition salts were not produced.

### Experimental Example 1

### Crystallinity/Hygroscopicity Test of Acid Addition Salts of Udenafil

The acid addition salts of Udenafil obtained in Examples 1 to 6 and Comparative Examples were tested for crystallinity and hygroscopicity as follows.

1. Crystallinity Test:

1) detecting whether the crystal is formed in the reaction solution, and

2) crystallization: detecting whether crystallization occurs after dissolving acid addition salt of Udenafil in a suitable solvent.

2. Hygroscopicity Test:

The change in weight is measured after the produced acid addition salt of Udenafil is exposed for 2 hours under the condition of room temperature and relative humidity 43%.

The test results are shown in Table 1

TABLE 1

| | | Crystal- | Crystal- | hygroscopicity |
|---|---|---|---|---|
| | | linity | linity | Hygroscopicity |
| | | test | test | (weight change |
| Organic acid | | 1-1) | 1-2) | ratio, %) |
| Example 1 | oxalic acid | ◯ | ◯ | 1.2 |
| Example 2 | benzenesulfonic acid | ◯ | ◯ | 1.4 |
| Example 3 | camphorsulfonic acid | ◯ | ◯ | 0.7 |
| Example 4 | cinnamic acid | ◯ | ◯ | 1.5 |
| Example 5 | adipic acid | ◯ | ◯ | 0.9 |
| Example 6 | cyclamic acid | ◯ | ◯ | 0.8 |
| Comparative | citric acid | X | X | — |
| Examples | succinic acid | X | X | — |
| | maleci acid | X | X | — |
| | malonic acid | X | X | — |
| | glutaric acid | X | X | — |
| | formic acid | X | X | — |
| | hippuric acid | X | X | — |
| | fumaric acid | X | X | — |
| | maleic acid | X | X | — |
| | aspartic acid | X | X | — |
| | glutamic acid | X | X | — |
| | tartaric acid | X | X | — |
| | lactic acid | X | X | — |

Note:
◯: Formation of crystal
X: No formation of crystal
—: The hygroscopicity test was not carried out, because the crystal was not formed.

As shown in Table 1, the acid addition salts of Udenafil according to Examples 1 to 6, where Udenafil was bonded to oxalic acid, benzenesulfonic acid, camphorsulfonic acid, cinnamic acid, adipic acid or cyclamic acid, showed crystallinity and low hygroscopicity when to expose to the high humidity, and thus the acid addition salts had excellent stability (particularly water stability) and could be easily formulated due to the crystallinity.

On the other hand, the acid addition salts of Udenafil obtained in Comparative Examples could not formed in a crystalline form, and thus was difficult to be formulated.

### Experimental Example 2

### Solubility Test

To test the solubility of the acid addition salts of Udenafil in distilled water, the following experiments were preformed.

Specifically, to test the solubility of the acid addition salt of Udenafil obtained Examples 1 to 6, the high performance liquid chromatography (HPLC) was performed. The test result was shown in Table 2.

TABLE 2

| | | First | second | third | Average of |
|---|---|---|---|---|---|
| | | measured | measured | measured | measured |
| | | peak area | peak area | peak area | peak area |
| | | No | No | No | No |
| Udebafil | | detection | detection | detection | detection |
| Example 1 | oxalate | 4,092,373 | 4,066,592 | 4,063,319 | 4,074,095 |
| Example 2 | Besylate | 59,985 | 59,985 | 58,993 | 59,654 |
| Example 3 | Camphor- | 7,141,140 | 7,127,027 | 7,158,961 | 7,142,473 |
| | sulfonate | | | | |
| Example 4 | Cinnamate | 806,024 | 812,746 | 824,937 | 814,569 |
| Example 6 | adipiate | 8,675,326 | 8,649,747 | 8,639,404 | 8,654,826 |
| Example 6 | cyclamate | 924,971 | 937,066 | 930,868 | 930,968 |

The analysis conditions for HPLC were as follows:

Detector: ultraviolet ray absorbance measured at wavelength 292 nm

Column: octadecyl silica gel C18 (4.6×150 mm, 5 μm)

Mobile phase: an aqueous solution of potassium dihydrogenphosphate (0.02M):acetonitrile=70:30

Flow rate: 1.0 ml/minute

sample volume: 10 μl

As shown in Table 2, Udenafil was not detected in the distilled water, but all of the acid addition salts of Udenafil in Table 2 were detected.

This result showed that Udenafil is hardly dissolved in the distilled water, but the specific acid addition salts of Udenafil of Examples 1 to 6 have excellent solubility in an aqueous solvent, especially in the distilled water. Particularly, Udenafil salts of adipic acid, camphorsulfonic acid and oxalic acid have remarkably excellent solubility in the distilled water.

What is claimed is:

1. An acid addition salt of Udenafil in which Udenafil is bonded to an organic acid selected from the group consisting of oxalic acid, benzenesulfonic acid, camphorsulfonic acid, cinnamic acid, adipic acid and cyclamic acid.

2. The acid addition salt of Udenafil according to claim 1, wherein the acid addition salt of Udenafil is represented by Chemical formula 2: 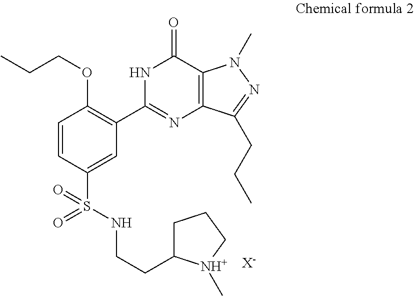
Wherein, X⁻ is

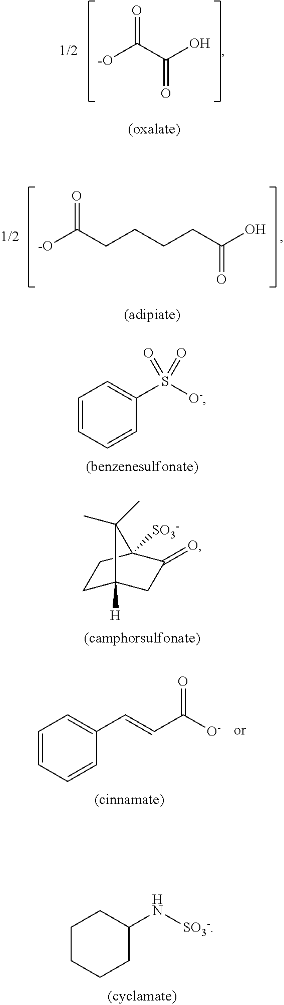

3. A method of preparing an acid addition salt of Udenafil comprising a step of reacting Udenafil with an organic acid selected from the group of consisting of oxalic acid, benzenesulfonic acid, camphorsulfonic acid, cinnamic acid, adipic acid and cyclamic acid.

4. The method of preparing an acid addition salt of Udenafil according to claim 3, wherein the Udenafil is reacted with the organic acid in at least one solvent selected from the group consisting of acetone, ethylacetate, methanol, ethanol, tetrahydrofuran and acetonitrile.

5. The method of preparing an acid addition salt of Udenafil according to claim 3, wherein the mixing ratio of the organic acid to the Udenafil is 0.95 to 1.1 equivalents.

6. A pharmaceutical composition comprising an acid addition salt of Udenafil of claim 1 as an active ingredient for use of prevention or treatment of impotence, portal hypertension, pulmonary hypertension, benign prostatic hyperplasia associated with lower urinary tract symptom, heart failure or chronic obstructive pulmonary disease.

7. The pharmaceutical composition according to claim 6, wherein the acid addition salt of Udenafil is contained in an amount of 10 to 100 mg.
